(19) United States
(12) Patent Application Publication
(10) Pub. No.: US 2010/0076069 A1
(43) Pub. Date: Mar. 25, 2010

(54) FORSKOLIN DOSING REGIMEN IN OPEN ANGLE GLAUCOMA AND PATIENT COMPLIANCE METHODS THEREOF

(76) Inventor: Muhammed Majeed, Piscataway, NJ (US)

Correspondence Address: SABINSA CORPORATION, 70 ETHEL ROAD WEST, UNIT 6, PISCATAWAY, NJ, 08854, US

(21) Appl. No.: 12/235,222

(22) Filed: Sep. 22, 2008

(51) Int. Cl. A61K 31/352; A61P 27/02

(52) U.S. Cl. 514/455

(57) ABSTRACT

Disclosed is an optimal dosing regimen for forskolin to ensure the most optimal pharmacokinetic/pharmacodynamic responses desired in the therapeutic management of open angle glaucoma. The optimal dosing regimen involves the ocular application of 2% forskolin solubilized using randomly-methylated beta-cyclodextrins one drop once a day to patients requiring therapy for open angle glaucoma. Also disclosed are methods of promoting patient compliance, in particular geriatric and pediatric patient compliance during the therapeutic management of glaucoma using an optimal dosing regimen for forskolin to ensure the best pharmacokinetic/pharmacodynamic effects, devoid of systemic side-effects encountered with the currently used beta-blockers or prostaglandins.

### BACKGROUND OF THE INVENTION

1. Field of the Invention

The present invention in general relates to ocular hypotensive agents. More specifically, the present invention relates to optimized forskolin dosage regimens comprising 2% forskolin solubilized using randomly-methylated beta-cyclodextrins for use as an ocular application one drop once a day in patients requiring therapy for open angle glaucoma and methods thereof to promote patient compliance during the therapeutic management of open angle glaucoma.

2. Description of Prior Art

Glaucoma is an eye disorder characterized by increased intraocular pressure, excavation of the optic nerve head and gradual loss of the visual field. Open angle glaucoma is the leading cause of blindness among adults in the United States and is particularly dangerous because it can progress gradually and go unnoticed for years. There are five main types of drug used in the treatment of glaucoma—miotics, sympathomimetics, beta-blockers, carbonic anhydrase inhibitors, and latanoprost (XALATAN). Table A

TABLE A

| Anti-glaucoma agent | Specific examples |
|---|---|
| MIOTICS | Carbachol (ISOPTO CARBACHOL); Pilocarpine (ISOPTO |
| | CARPINE) |
| SYMPATHOMIMETICS | Adrenaline/epinephrine (EPPY, SIMPLENE); Brimonidine tartrate |
| | (ALPHAGAN - Black triangle); Dipivefrine hydrochloride |
| | (PROPINE); Guanethidine monosulphate (GANDA) |
| BETA-BLOCKERS | Betaxolol hydrochloride (BETOPTIC); Carteolol hydrochloride |
| | (TEOPTIC); Levobunolol hydrochloride (BETAGAN); |
| | Metipranolol; Timolol maleate (TIMOPTOL) |
| CARBONIC ANHYDRASE | Acetazolamide (DIAMOX); Brinzolamide (AZOPT - Black |
| INHIBITORS | triangle); Dorzolamide (TRUSOPT) |
| PROSTAGLANDIN | Latanoprost (XALATAN) |
| ANALOGUE | |

Prostaglandin analogs and derivatives as ocular hypotensive agents have been discussed in numerous patent references including U.S. Pat. No. 5,151,444, U.S. Pat. No. 5,166,178, U.S. Pat. No. 5,194,429, U.S. Pat. No. 5,422,368, U.S. Pat. No. 5,849,791 and U.S. Pat. No. 6,030,999. Latanoprost (XALATAN), a prostaglandin analogue (PGF2a), is a commonly used antiglaucoma medication.

The systemic side effects of PGF2a has been indicated in
A. Lai J, Jin H, Yang R, Winer J, Li W, Yen R et al. Prostagladin F2 induces cardiac myocyte hypertrophy in vitro and cardiac growth in vivo. Am J Physiol 1996; 271: 2197-2208;
B. Rao T S, Seth S D, Nayar U, Manchanda S C. The vagal involvement in the antiarrhythmic and arrhythmogenic effects of prostaglandin F2 alpha on ouabain-induced cardiac arrhythmias in cats. Life Sci 1987; 41: 2363-2372.
C. Shigematsu S, Niwa H, Saikawa T. Vasospastic angina induced by prostaglandin F2 alpha. Br Heart J 1993; 69: 364-365.

The documented side effects of using XALATAN include
A. Serious side effects including redness, swelling, itching, or pain in or around your eye; oozing or discharge from the eye; increased sensitivity to light; vision changes; or chest pain.
B. Less serious side effects may include cold symptoms such as stuffy nose, sneezing, sore throat; headache, dizziness; mild eye discomfort; blurred vision; feeling of foreign substances in the eye; dry or watery eyes; or stinging or burning of the eyes after using the drops.

Beta blockers on the other hand can trigger asthma attacks, fainting episodes, and cause central nervous system side effects such as depression and decreased sexual function.

0.09% to 6% diterpenes such as forskolin, isoforskolin and deacetylforskolin solubilized using randomly methylated beta cyclodextrins and their use in reducing intraocular pressure in glaucoma has been indicated in U.S. Pat. No. 6,960,300 B2 to the inventors of the present invention. With practically no peripheral and systemic side effects, the present inventors chose to device suitable forskolin based dosage regimens to improve patient compliance during the therapeutic management of open angle glaucoma.

Accordingly, it is the principle object of the present invention to develop optimized forskolin based dosage regimens for the most optimal pharmacodynamic/pharmacokinetic response desired in the therapeutic management of open angle glaucoma, said dosage regimen comprising 2% forskolin solubilized using randomly-methylated beta-cyclodextrins for use as an ocular application one drop once a day in patients requiring therapy for glaucoma.

It is another object of the present invention to develop a method to promote patient compliance during the therapeutic management of open angle glaucoma, said method involving a specific pharmaceutical dosage regimen comprising 2% forskolin solubilized using randomly-methylated beta-cyclodextrins for use as an ocular application one drop once a day requiring therapy for open angle glaucoma.

It is yet another object of the present invention to develop pharmaceutical dosage regimens of forskolin which are designed to increase patient compliance during forskolin therapy for glaucoma, said dosage regimens comprising 2% forskolin solubilized using randomly-methylated beta-cyclodextrins for use as an ocular application one drop once a day in patients requiring therapy for open angle glaucoma.

Further, the invention seeks to disclose a method to treat open angle glaucoma, said method comprising the ocular administration in patients in need of such treatment, an optimized forskolin dosage regimen comprising 2% forskolin solubilized using randomly-methylated beta-cyclodextrins one drop once a day.

The present invention fulfills the aforesaid objectives and provides further related advantages.

### SUMMARY OF THE INVENTION

The present invention discloses an optimal dosing regimen for forskolin to ensure the most optimal pharmacokinetic/pharmacodynamic responses desired in the therapeutic management of glaucoma. The optimal dosing regimen involves the application of 2% forskolin solubilized using randomly-methylated beta-cyclodextrins one drop once a day in patients requiring therapy for open angle glaucoma. Also disclosed are methods of promoting patient compliance, in particular geriatric and pediatric patient compliance during the therapeutic management of glaucoma using an optimal dosing regimen for forskolin to ensure the best pharmacokinetic/pharmacodynamic responses, devoid of systemic side-effects encountered with the currently used beta-blockers or prostaglandins.

The advantages of the present invention include:
1. Optimized forskolin dosage regimens for the most optimal pharmacodynamic/pharmacokinetic responses desired in the therapeutic management of open angle glaucoma, said dosage regimen comprising 2% forskolin solubilized using randomly-methylated beta-cyclodextrins for use as an ocular application one drop once a day in patients requiring therapy for glaucoma.
2. A method to promote patient compliance during the therapeutic management of open angle glaucoma, said method involving a specific pharmaceutical dosage regimen comprising 2% w/w forskolin solubilized using randomly-methylated beta-cyclodextrins for use as an ocular application one drop once a day in patients requiring therapy for open angle glaucoma.
3. Pharmaceutical dosage regimens of forskolin which are designed to increase patient compliance during forskolin therapy for glaucoma, said dosage regimens comprising 2% w/w forskolin solubilized using randomly-methylated beta-cyclodextrins for use as an ocular application one drop once a day in patients requiring therapy for open angle glaucoma.
4. A method to treat open angle glaucoma, said method comprising the ocular administration in patients in need of such treatment, an optimized forskolin dosage regimen comprising 2% forskolin solubilized using randomly-methylated beta-cyclodextrins one drop once a day.
5. Optimized forskolin dosage regimens for the best pharmacodynamic/pharmacokinetic response desired in the therapeutic management of open angle glaucoma with no serious systemic side effects.

Other features and advantages of the present invention will become apparent from the following more detailed description, taken in conjunction with the accompanying drawings, which illustrate, by way of example, the principle of the invention.

### DESCRIPTION OF THE DRAWINGS

FIG. 1 is a graphical representation of the baseline intra ocular pressure in patients suffering from open angle glaucoma

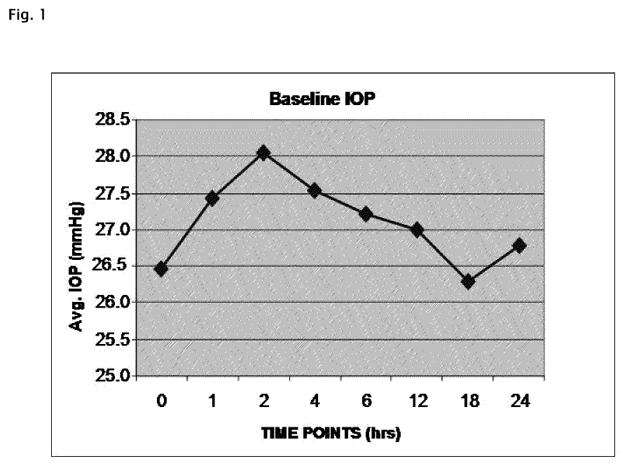

FIG. 2 is a graphical representation of the fall in intraocular pressure in patients undergoing therapy with 1% w/w forskolin (two drops once a day—9.00 pm instillation)

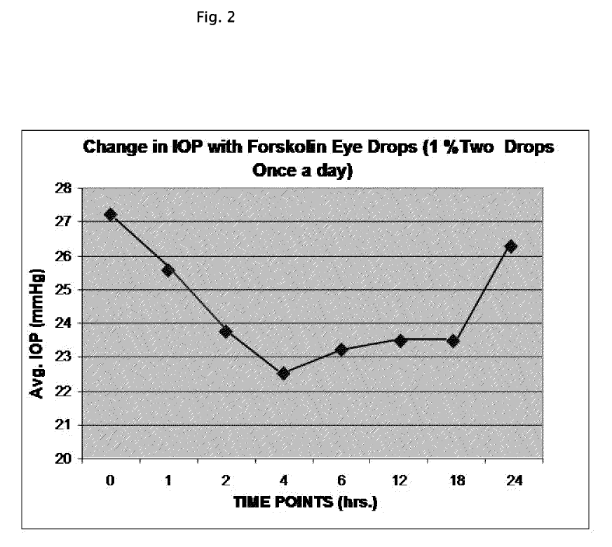

FIG. 3 is a graphical representation of the fall in intraocular pressure in patients who are undergoing therapy with 2% w/w Forskolin (one drop once a day—9:00 PM instillation)

### DETAILED DESCRIPTION OF THE PREFERRED EMBODIMENT

The present inventors had developed 1 % w/w Ophthalmic solution of forskolin, 3 times a day for treatment of Glaucoma. Three times a day regimen was found to be a poor compliance with the patients. Hence, it was decided to try 2% w/w solution once a day. Therefore, instead of one drop application of 1% w/w solution, it was decided to make the dose as two drops at a time. Thus, the eye was getting equivalent to 2% w/w of one drop. This was not successful. Concurrently, a 2% w/w solution was made and one drop once a day was the regimen. The 2% solution dosed was effective for more than 18 hours after application. This result was unexpected because two drops with 1% w/w concentration of forskolin ophthalmic solution also was delivering into the eye, the exact same equivalent quantity of the active drug forskolin as one drop of 2% w/w solution but, still the latter was surprisingly more effective. Additionally application of one drop of 2% w/w solution (option 1) vs two drops of 1% w/w solution (option 2), had the following unexpected benefits. The initial elevated values of ocular pressure was restored to normal values much more quickly in the case of option 1 even though as stated earlier the eye was in contact with the same quantity/amount of active drug in both options. Also the maintenance of ocular pressure in the normal range was longer in the case of option 1

In the most preferred embodiment, the present invention relates to optimized forskolin dosage regimens for the best pharmacodynamic/pharmacokinetic response desired in the therapeutic management of open angle glaucoma glaucoma, said dosage regimen comprising 2% forskolin solubilized using randomly-methylated beta-cyclodextrins for use as an ocular application once a day in patients requiring therapy for open angle glaucoma.

In an additional embodiment, the present invention discloses a method to promote patient compliance during the therapeutic management of open angle glaucoma, said method involving a specific pharmaceutical dosage regimen comprising 2% forskolin solubilized using randomly-methylated beta-cyclodextrins for use as an ocular application once a day in patients requiring therapy for open angle glaucoma. This is particularly relevant in geriatric patients who would prefer to do away with multiple applications in the eye during a single day and associated physical stress. This is also relevant in pediatric patients.

In a further additional embodiment, the present invention relates to pharmaceutical dosage regimens of forskolin which are designed to increase patient compliance during forskolin therapy for glaucoma, said dosage regimens comprising 2% forskolin solubilized using randomly-methylated beta-cyclodextrins for use as an ocular application once a day one time application in patients requiring therapy for open angle glaucoma.

In another additional embodiment, the present invention also relates to a method to treat open angle glaucoma, said method comprising the ocular administration in patients in need of such treatment, an optimized forskolin dosage regimen comprising 2% w/w forskolin solubilized using randomly-methylated beta-cyclodextrins once a day.

The most preferred embodiments of the present invention are further elucidated using specific examples herein below.

### EXAMPLE I

### Clinical Studies

### Study Protocol

a) No. of subjects: 6

b) Dosage Regimen:
I. Forskolin Eye drops (1% w/w)—Two Drops once a day (instillation at 9.00pm)
II. Forskolin Eye Drops (2.0% w/w)—ONE DROP ONCE A day (instillation at 9:00 PM)

c) Intraocular pressure (IOP) recorded at 0, 1, 2, 4, 6, 12, 18, 24 hrs.

d) Wash out-period of 7 days between the different treatment regimens

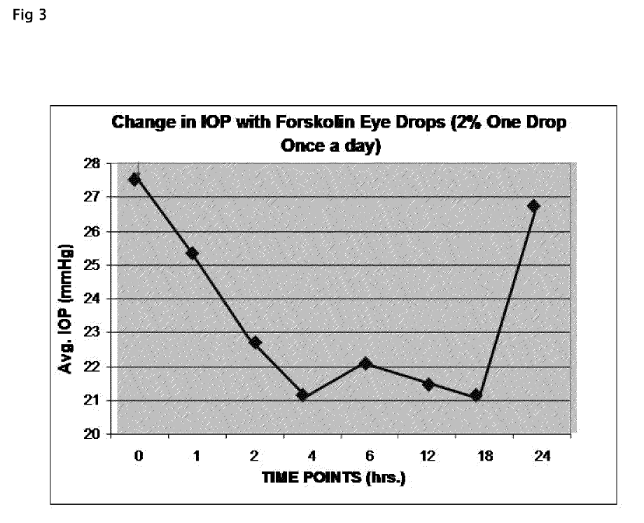

(FIG. 3) 2% forskolin ocular application one drop once a day at 9 PM showed immediate effect in bringing down the average IOP mm/Hg from a value of 28.0 to 21.0 in 4 hours with the effect sustaining till about 18 hours after instillation without any systemic side effects.

The advantages of the present invention include:
1. Optimized forskolin dosage regimens for the most optimal pharmacodynamic/pharmacokinetic responses desired in the therapeutic management of open angle glaucoma, said dosage regimen comprising 2% w/w forskolin solubilized using randomly-methylated beta-cyclodextrins for use as an ocular application one drop once a day for patients requiring therapy for glaucoma.
2. A method to promote patient compliance during the therapeutic management of open angle glaucoma, said method involving a specific pharmaceutical dosage regimen comprising 2% w/w forskolin solubilized using randomly-methylated beta-cyclodextrins for use as an ocular application one drop once a day in patients requiring therapy for open angle glaucoma.
3. Pharmaceutical dosage regimens of forskolin which are designed to increase patient compliance during forskolin therapy for glaucoma, said dosage regimens comprising 2% w/w forskolin solubilized using randomly-methylated beta-cyclodextrins for use as an ocular application one drop once a day in patients requiring therapy for open angle glaucoma.
4. A method to treat open angle glaucoma, said method comprising the ocular administration in patients in need of such treatment, an optimized forskolin dosage regimen comprising 2% w/w forskolin solubilized using randomly-methylated beta-cyclodextrins one drop once a day.
5. Optimized forskolin dosage regimens for the best pharmacodynamic/pharmacokinetic response desired in the therapeutic management of open angle glaucoma with no serious systemic side effects.

While the invention has been described with reference to a preferred embodiment, it is to be clearly understood by those skilled in the art that the invention is not limited thereto. Rather, the scope of the invention is to be interpreted only in conjunction with the appended claims.

We claim:

1. Optimized forskolin dosage regimens for the most optimal pharmacodynamic/pharmacokinetic responses desired in the therapeutic management of open angle glaucoma glaucoma, said dosage regimen comprising 2% w/w forskolin solubilized using randomly-methylated beta-cyclodextrins for use as an ocular application one drop once a day requiring therapy for open angle glaucoma.

2. A method to promote patient compliance during the therapeutic management of open angle glaucoma, said method involving a specific pharmaceutical dosage regimen comprising 2% w/w forskolin solubilized using randomly-methylated beta-cyclodextrins for use as an ocular application one drop once a day in patients requiring therapy for open angle glaucoma.

3. Pharmaceutical dosage regimens of forskolin which are designed to increase patient compliance during forskolin therapy for glaucoma, said dosage regimens comprising 2% w/w forskolin solubilized using randomly-methylated beta-cyclodextrins for use as an ocular application one drop once a day in patients requiring therapy for open angle glaucoma.

4. The dosage regimens as in any one of claims 1, 2 or 3, in which said dosage regimens show enhanced efficacy in lowering the intraocular pressure without any peripheral/systemic side effects in comparison to beta-blockers or prostaglandins.

5. A method to treat open angle glaucoma, said method comprising the ocular administration in patients in need of such treatment, an optimized forskolin dosage regimen comprising 2% w/w forskolin solubilized using randomly-methylated beta-cyclodextrins one drop once a day.
